(19) United States
(12) Patent Application Publication
(10) Pub. No.: US 2025/0139768 A1
(43) Pub. Date: May 1, 2025

(54) METHOD FOR ACQUIRING CLASSIFICATION MODEL, METHOD FOR DETERMINING EXPRESSION CATEGORY, APPARATUS, DEVICE AND MEDIUM

(71) Applicant: BOE Technology Group Co., Ltd., Beijing (CN)

(72) Inventors: Yulan Hu, Beijing (CN); Cuifang Zhang, Beijing (CN)

(73) Assignee: BOE Technology Group Co., Ltd., Beijing (CN)

(21) Appl. No.: 18/694,188

(22) PCT Filed: Jul. 31, 2023
(86) PCT No.: PCT/CN2023/110354

§ 371 Date: Mar. 21, 2024

(30) Foreign Application Priority Data
Sep. 19, 2022 (CN) 202211140564.6

(51) Int. Cl. G06T 7/00; G06V 10/44; G06V 10/54; G06V 10/764; G06V 10/80; G16H 30/40

(52) U.S. Cl. CPC G06T 7/0012; G06V 10/44; G06V 10/54; G06V 10/764; G06V 10/806; G16H 30/40; G06T 2207/20081; G06T 2207/30016; G06T 2207/30096; G06V 2201/07

(57) ABSTRACT

A method and an apparatus for acquiring a classification model, a method and an apparatus for determining an expression category, a device, and a medium are provided. The method for acquiring the classification model includes: for a tumor region of a sample object, acquiring a plurality of radiomics features and a plurality of voxel features of the tumor region; screening the plurality of radiomics features based on a first screening factor to obtain a plurality of radiomics feature samples; and screening the plurality of voxel features based on a second screening factor to obtain a plurality of voxel feature samples; wherein each of the first screening factor and the second screening factor includes an expression category label of a target gene of the sample object; constructing training samples based on the plurality of radiomics feature samples and the plurality of voxel feature samples.

### CROSS REFERENCE TO RELATED APPLICATIONS

The present disclosure claims the priority of the Chinese patent application filed on Sep. 19, 2022 before the Chinese Patent Office with the application number of 202211140564.6 and the title of “METHOD FOR ACQUIRING CLASSIFICATION MODEL, METHOD FOR DETERMINING EXPRESSION CATEGORY, APPARATUS, DEVICE AND MEDIUM”, which is incorporated herein in its entirety by reference.

### TECHNICAL FIELD

The present disclosure relates to the technical field of data processing and, more particularly, to a method and an apparatus for acquiring a classification model, a method and an apparatus for determining an expression category, a device, and a medium.

### BACKGROUND

For common tumors, especially neuroglioma, there are many molecular markers of the tumors and signal pathways involved, such as isocitrate dehydrogenase (IDH) mutation, O6-methylguanine DNA methyltransferase (MGMT) promoter methylation, chromosome 1p/19q deletion, epidermal growth factor receptor (EGFR) amplification, telomerase reverse tranase (TERT) gene promoter (TERTp) mutation, H3F3A mutation, a Notch pathway, miRNAs, etc.

An expression type of the above-mentioned gene may be used as a physiological parameter for detection and prognosis of tumors.

### SUMMARY

A method for acquiring a classification model is provided by the present disclosure, wherein the method includes:
for a tumor region of a sample object, acquiring a plurality of radiomics features and a plurality of voxel features of the tumor region;
screening the plurality of radiomics features based on a first screening factor to obtain a plurality of radiomics feature samples; and screening the plurality of voxel features based on a second screening factor to obtain a plurality of voxel feature samples; wherein each of the first screening factor and the second screening factor includes an expression category label of a target gene of the sample object;
constructing training samples based on the plurality of radiomics feature samples and the plurality of voxel feature samples; and
training a preset model by taking the training samples as inputs to obtain the classification model, wherein the classification model is configured to predict an expression category of the target gene.

In an optional embodiment, acquiring the plurality of radiomics features of the tumor region includes:
extracting a first subregion image belonging to a tumor non-enhancement region, a second subregion image belonging to a tumor enhancement region and a third subregion image belonging to a peritumoral edema region from image samples of the tumor region; and
performing feature extraction on the first subregion image, the second subregion image and the third subregion image, respectively, to obtain the plurality of radiomics features.

In an optional embodiment, acquiring the plurality of radiomics features of the tumor region includes:
acquiring a plurality of types of image samples of the tumor region, wherein the plurality of types include a T1-weighted type, a T2-weighted type, a contrast-enhanced T1-weighted type and a T2 fluid-attenuated inversion recovery type;
performing feature extraction on each of the plurality of types of image samples, respectively; and
combining the radiomics features respectively corresponding to each of the plurality of types of extracted image samples to obtain the plurality of radiomics features.

In an optional embodiment, the tumor region is a glioma region in a brain, and the method further includes:
determining position information corresponding to the glioma region based on an image sample of the glioma region;
acquiring position features corresponding to the position information; wherein the position information includes a brain region to which the glioma region belongs, and/or position coordinates of the glioma region in the brain; and
constructing the training samples based on the plurality of radiomics feature samples and the plurality of voxel feature samples includes:
constructing the training samples based on the position features, the plurality of radiomics feature samples and the plurality of voxel feature samples.

In an optional embodiment, the first screening factor includes the expression category label and a tumor grading label of the tumor region; and screening the plurality of radiomics features based on the first screening factor to obtain the plurality of radiomics feature samples includes:
screening the plurality of radiomics features based on a first relation value between each of the plurality of radiomics features and the expression category label to obtain a plurality of first radiomics features; wherein the first relation value is configured to represent a degree of correlation between each of the plurality of radiomics features and mutation of the target gene;
screening the plurality of radiomics features based on a second relation value between each of the plurality of radiomics features and the tumor grading label to obtain a plurality of second radiomics features; wherein the second relation value is configured to represent a degree of correlation between each of the plurality of radiomics features and a tumor grade; and
de-duplicating the plurality of first radiomics features and the plurality of second radiomics features to obtain the plurality of radiomics feature samples.

In an optional embodiment, a plurality of sample objects are included, and the method further includes:
for all radiomics features included by all the sample objects, screening all the plurality of radiomics features based on a third screening factor to obtain complementary radiomics feature samples; wherein the third screening factor includes clinical data respectively corresponding to the plurality of sample objects; and
constructing the training samples based on the plurality of radiomics feature samples and the plurality of voxel feature samples includes:
constructing the training samples based on the plurality of radiomics feature samples, the plurality of voxel feature samples, and the plurality of complementary radiomics feature samples.

In an optional embodiment, for all radiomics features included by all the sample objects, screening all the plurality of radiomics features based on the third screening factor to obtain the complementary radiomics feature samples includes:
acquiring a radiomics feature matrix and a clinical data matrix; wherein the radiomics feature matrix includes the plurality of radiomics features respectively corresponding to the plurality of sample objects, and the clinical data matrix includes the clinical data respectively corresponding to the plurality of sample objects;
acquiring a mutual information coefficient matrix based on the radiomics feature matrix and the clinical data matrix, wherein the mutual information coefficient matrix includes a mutual information coefficient between each of the plurality of radiomics features and the clinical data, and the mutual information coefficient is configured to represent a degree of correlation between each of the plurality of radiomics features and the clinical data; and
screening, based on the mutual information coefficient matrix, all the plurality of radiomics features included by the radiomics feature matrix to obtain the plurality of complementary radiomics feature samples.

In an optional embodiment, the second screening factor includes the expression category label, and screening the plurality of voxel features based on the second screening factor to obtain the plurality of voxel feature samples includes:
acquiring a variance of each of the plurality of voxel features, and retaining voxel features of which variances are greater than a first variance threshold to obtain a plurality of candidate voxel features; and
taking the expression category label as a predication label and taking the plurality of candidate voxel features as inputs, screening the plurality of voxel feature samples from the plurality of candidate voxel features by using a linear regression model.

In an optional embodiment, before screening the plurality of radiomics features based on the first screening factor to obtain the plurality of radiomics feature samples, the method further includes:
determining a variance corresponding to each of the plurality of the radiomics features, and retaining radiomics features of which variances are greater than a second variance threshold to obtain a plurality of candidate radiomics features; and
screening the plurality of radiomics features based on the first screening factor to obtain the plurality of radiomics feature samples includes:
screening the plurality of candidate radiomics features based on the first screening factor to obtain the plurality of radiomics feature samples.

In an optional embodiment, acquiring the plurality of radiomics features of the tumor region includes:
acquiring wavelet images and laplacian of gaussian (LoG) images of the image samples of the tumor region;
performing multi-scale feature extraction on the image samples of the tumor region, the wavelet images and the LoG images, respectively, to obtain first-order statistics features, texture features and morphological features of the tumor region; and
combining the first-order statistics features, the texture features and the morphological features of the tumor region to obtain the plurality of radiomics features.

In an optional embodiment, training the preset model by taking the training samples as the inputs to obtain the classification model includes:
inputting the training samples to the classification model to obtain a predicted expression category, outputted by the classification model, of the target gene;
determining a loss value of the classification model based on the predicted expression category and the expression category label;
updating parameters of the classification model based on the loss value; and
taking a classification model satisfying a training ending condition as the classification model, wherein the training ending condition is that the classification model converges or reaches a time quantity of preset updating.

A method for determining an expression category of a target gene is further provided by the present disclosure, wherein the method includes:
acquiring a plurality of target radiomics features and a plurality of target voxel features of a tumor region of a to-be-tested object;
inputting the plurality of target radiomics features and the plurality of target voxel features to the classification model, wherein the classification model is obtained according to the method for acquiring the classification model; and
determining an expression category of a target gene of the to-be-tested object based on an output of the classification model.

In an optional embodiment, after acquiring the plurality of target radiomics features and the plurality of target voxel features of the tumor region of the to-be-tested object, the method further includes:
determining a variance corresponding to each of the plurality of target voxel features, and retaining target voxel features of which variances are greater than a first variance threshold; and
determining a variance corresponding to each of the plurality of target radiomics features, and retaining target radiomics features of which variances are greater than a second variance threshold;
inputting the plurality of target radiomics features and the plurality of target voxel features to the classification model includes:
inputting the retained target radiomics features and the retained target voxel features to the classification model.

In an optional embodiment, after acquiring the plurality of target radiomics features and
the plurality of target voxel features of the tumor region of the to-be-tested object, the method further includes:
acquiring a fourth screening factor corresponding to the to-be-tested object, wherein the fourth screening factor includes clinical data and/or tumor grading data of the to-be-tested object;
screening the plurality of target radiomics features based on the fourth screening factor; and
inputting the plurality of target radiomics features and the plurality of target voxel features to the classification model includes:
inputting the plurality of screened target radiomics features and the plurality of screened target voxel features to the classification model.

In an optional embodiment, the fourth screening factor includes the clinical data and the tumor grading data; and screening the plurality of target radiomics features based on the fourth screening factor includes:
determining a third relation value between each of the target radiomics features and a tumor grading label and a mutual information coefficient between each of the plurality of target radiomics features and the clinical data;
screening the plurality of target radiomics features based on the third relation value, and screening the plurality of target radiomics features based on the mutual information coefficient; and
de-duplicating the target radiomics features screened based on the third relation value and the target radiomics features screened based on the mutual information coefficient to obtain the screened target radiomics features.

An apparatus for acquiring a classification model is further provided by the present disclosure, wherein the apparatus includes:
a feature acquisition module configured to, for a tumor region of a sample object, acquire a plurality of radiomics features and a plurality of voxel features of the tumor region;
a feature selection module configured to screen the plurality of radiomics features based on a first screening factor to obtain a plurality of radiomics feature samples; and screen the plurality of voxel features based on a second screening factor to obtain a plurality of voxel feature samples; wherein each of the first screening factor and the second screening factor includes an expression category label of a target gene of the sample object;
a sample construction module configured to construct training samples based on the plurality of radiomics feature samples and the plurality of voxel feature samples; and
a model training module configured to train a preset model by taking the training samples as inputs to obtain the classification model, wherein the classification model is configured to predict an expression category of the target gene.

An apparatus for determining an expression category of a target gene is further provided by the present disclosure, wherein the apparatus includes:
a feature acquisition module configured to acquire a plurality of target radiomics features and a plurality of target voxel features of a tumor region of a to-be-tested object;
a feature input module configured to input the plurality of target radiomics features and the plurality of target voxel features to the classification model, wherein the classification model is obtained according to the method for acquiring the classification model; and
a category determination module configured to determine an expression category of a target gene of the to-be-tested object based on an output of the classification model.

An electronic device is further provided by the present disclosure, including a memory, a processor, and a computer program stored in the memory and capable of running on the processor, wherein the processor, when being executed, implements the method for acquiring the classification model, or when being executed, implements the method for determining the expression category of the target gene.

A computer-readable storage medium is further provided by the present disclosure, wherein a computer program stored thereon enables a processor, when being executed, to implement the method for acquiring the classification model, or enables the processor, when being executed, to implement the method for determining the expression category of the target gene.

By adopting the method for acquiring the classification model in the present disclosure, for a tumor region of a sample object, a plurality of radiomics features and a plurality of voxel features of the tumor region may be acquired; the plurality of radiomics features are screened based on a first screening factor to obtain a plurality of radiomics feature samples; and the plurality of voxel features are screened based on a second screening factor to obtain a plurality of voxel feature samples; training samples are constructed based on the plurality of radiomics feature samples and the plurality of voxel feature samples; and next, a preset model is trained by taking the training samples as inputs to obtain the classification model, wherein the classification model is configured to predict an expression category of a target gene.

On one hand, since each of the first screening factor and the second screening factor includes an expression category label of the target gene of the sample object, the expression category label may represent the expression category of the target gene, such as a mutation category and a deletion state, in this way, the radiomics feature samples and the voxel feature samples which are closely correlated to the expression category of the target gene may be screened by taking the expression category of the target gene as a physiological parameter, and then, the preset model is trained by taking the screened radiomics feature samples and voxel feature samples as the training samples, so that the classification model can learn the correlation between a morphological characteristic of the tumor region and the expression category of the target gene, the interpretability of the classification model is improved, and then, the accuracy of predicting the expression category of the target gene based on an image of the tumor region is improved.

On the other hand, since the training samples not only include the radiomics features of the tumor region, but also include the voxel features of the tumor region, wherein the radiomics features may reflect three-dimensional features such as a texture and a shape of the tumor region, and the voxel features may reflect three-dimensional features such as a spatial three-dimensional morphology of the tumor region, so that the richness of the training samples can be improved, and then, the accuracy of the classification model is improved.

The above description is only a summary of technical schemes of the present disclosure, which can be implemented according to contents of the specification in order to better understand technical means of the present disclosure; and in order to make above and other objects, features and advantages of the present disclosure more obvious and understandable, detailed description of the present disclosure is particularly provided in the following.

### BRIEF DESCRIPTION OF THE DRAWINGS

In order to more clearly illustrate the technical solutions of the embodiments of the present disclosure or the prior art, the figures that are required to describe the embodiments or the prior art may be briefly introduced below. Apparently, the figures that are described below are embodiments of the present disclosure, and a person skilled in the art can obtain other figures according to these figures without paying creative work. It should be noted that the proportions in the drawings are only indicative and do not represent actual proportions.

FIG. 1 schematically shows a schematic diagram of an overall flow of a process that a classification model is acquired;

FIG. 2 schematically shows a flow diagram of steps of a method for acquiring a classification model;

FIG. 3 schematically shows a schematic diagram of a process that radiomics features are completely extracted in the present disclosure;

FIG. 4 schematically shows a flow diagram of steps for screening radiomics features based on clinical data;

FIG. 5 schematically shows a flow diagram of steps of a method for determining an expression category of a target gene;

FIG. 6 schematically shows a schematic diagram of a structural framework of an apparatus for acquiring a classification model;

FIG. 7 schematically shows a schematic diagram of a structural framework of an apparatus for determining an expression category of a target gene; and

FIG. 8 schematically shows a structural block diagram of an electronic device in the present disclosure.

### DETAILED DESCRIPTION OF THE EMBODIMENTS

In order to make purposes, technical schemes and advantages of embodiments of this disclosure more clearer, the technical schemes in the embodiments of this disclosure will be described clearly and completely with reference to the drawings in the embodiments of this disclosure; and it is obvious that the described embodiments are part of the embodiments of this disclosure, but not all of them. On a basis of the embodiments in this disclosure, all other embodiments obtained by the ordinary skilled in the art without paying creative effort are within a protection scope of this disclosure.

For neuroglioma (brain glioma), a subtype (mutant type/wild type) of an IDH genetype of brain glioma, a deletion state (deletion/undeletion) of a 1p/19q chromosome and a methylation state (methylation/unmethylation) of MGMT, which serve as markers and signal transduction pathways, take part in the occurrence and development of the glioma, and have a significant effect on the proliferation, metastasis and invasion of the glioma.

Taking a telomerase reverse tranase (TERT) gene as an example, it is one of important genes of an encoding telomerase complex, the TERT gene has no transcriptional activity in most of non-tumor cells, but generates TERT gene mutation, such as promoter mutation, gene translocation and DNA amplification, in seventy-three percent of tumors. That is to say, there is certain correlation between an expression category of the above-mentioned gene and a tumor.

At present, chemotherapy is still one of important means for treating neuroglioma. However, side effects brought by chemotherapy drugs are increasingly prominent, and the effect is not ideal. An expression type of a gene is generally required to be invasively detected, which brings greater pain to patients. Therefore, prediction for a biological index state of the above-mentioned gene can help to provide reference parameters for the treatment of brain glioma to develop treatment plans.

In order to realize that an expression category of a target gene is detected, to provide a reliable physiological parameter for precisely detecting a tumor, a method for determining an expression category of a target gene based on radiomics and a neural network is provided by the present disclosure. The method can non-invasively realize the detection for the target gene, and has the main core concept that screened radiomics features are obtained by utilizing radiomics features of a magnetic resonance (MR) segmented image and combining with a plurality of feature screening methods; next, voxel calculation is performed on a magnetic resonance imaging (MRI) brain image by using a voxel-based morphometry (VBM) method, and analysis for the correlation between a voxel and the target gene is performed on the obtained voxel features to obtain screened voxel features; and then, a classifier is trained based on the fusion of the radiomics features and the voxel features to obtain a classification model, wherein the classification model is used to predict an expression category of a to-be-predicted target gene.

The tumor referred to in the present disclosure may be a common tumor such as brain glioma, liver tumor, breast tumor, thyroid tumor, lung tumor and melanoma tumor. In the present disclosure, the brain glioma is mainly described as an example.

It should be noted that the present disclosure provides the method for determining the expression category of the target gene based on radiomics and a neural network, and therefore, the present disclosure aims at constructing a classification model configured for predicting an expression category of a target gene by means of the thought of machine learning. In order to ensure that the classification model has higher interpretability, a technical means for screening (analysis for the correlation between the voxel and the target gene) the radiomics features (screening the radiomics features in combination with the plurality of features) and voxel features is put forward, thereby the effectiveness of training samples is improved.

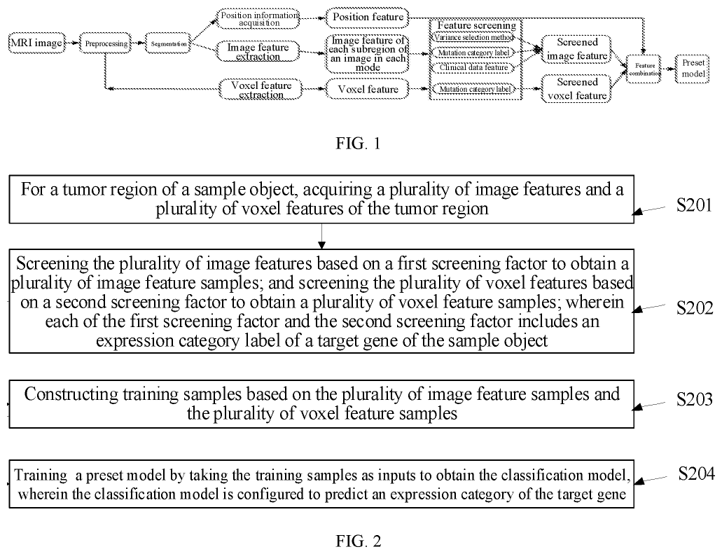

With reference to FIG. 1, a schematic diagram of an overall flow of a process that a classification model is acquired is shown. With reference to FIG. 1, a three-dimensional image including a tumor region may be segmented after being pre-processed to obtain an image of the tumor region; next, feature extraction is performed on the image of the tumor region to obtain radiomics features; and voxel feature calculation is performed on the pre-processed three-dimensional image to obtain the voxel features.

Then, feature screening is performed, the plurality of screening methods are performed on the radiomics features to obtain feature-screened radiomics features, and target gene correlation analysis is performed on the voxel features to obtain screened voxel features; and next, the screened voxel features and radiomics features are inputted to a classifier for training after being fused so as to be trained to obtain a classification model.

With reference to FIG. 2, a flow diagram of steps of a method for acquiring a classification model in the present disclosure is shown. As shown in FIG. 2, the method may specifically include the following steps:
step S201: for a tumor region of a sample object, a plurality of radiomics features and a plurality of voxel features of the tumor region are acquired.

In the present embodiment, the sample object may be referred to a tumor patient, wherein the plurality of radiomics features and the plurality of voxel features of the tumor region may be extracted from a magnetic resonance image of the tumor region, and the plurality of radiomics features may be obtained by performing feature extraction on a three-dimensional image of the tumor region.

In reality, the three-dimensional image is the magnetic resonance image. Since the tissue density of a human body is nonuniform, a medium may be divided into a plurality of small cubic blocks with relatively uniform density during scanning, such small cubic blocks are referred to as voxels which are basic units forming the three-dimensional image, and the smaller the voxels are, the clearer the image is.

The radiomics features may reflect two-dimensional features such as a slice texture and shape of the tumor region, and the voxel features may reflect three-dimensional features such as a spatial three-dimensional morphology of the tumor region. In this way, each-dimensional features of a tumor can be obtained to fully reflect morphological features of the tumor region and enrich feature information.

The process that the plurality of radiomics features are obtained may be described as follows:

image frames having different slice thicknesses (ranging from one to ten) and pixel spacings at the position of the tumor and generated by different MRI machines are resampled to a uniform slice thickness of one point zero and a pixel interval of [1, 1, 1] to obtain a three-dimensional image; and a processed image is obtained after the three-dimensional image is normalized by adopting a mean value and a standard deviation. The processed image is segmented to obtain an image of the tumor region, and next, feature extraction is performed on the image of the tumor region to obtain the plurality of radiomics features. During specific implementation, an image segmentation module realizes segmentation of the tumor region by adopting a UNet segmentation network model, and training inputs of the UNet segmentation network model include three-dimensional image data in four modes, which are labeled as segmented mask images.

The process that the plurality of voxel features are obtained may be described as follows:
taking the brain glioma as an example, image frames having different slice thicknesses (ranging from one to ten) and pixel spacings at the position of the tumor and generated by different MRI machines are resampled to a uniform slice thickness of one point zero and a pixel interval of [1,1,1] to obtain a three-dimensional image. The three-dimensional image is aligned to a T1-weighted template image at the position of the tumor, is then standardized to the space of the Montreal Neurological Institute, and is extracted by using an eight-millimeter full width at half maximum (FWHM) kernel to generate a gray matter density image including eight million nine hundred and twenty-eight thousand voxels, which a product of two hundred and forty, two hundred and forty, and one hundred and fifty-five, and the voxels are in sizes of 1*1*1 cubic millimeters. The present example is described by taking the brain glioma as an example, and other types of tumors may be described with reference to the same.
Step S202: the plurality of radiomics features are screened based on a first screening factor to obtain a plurality of radiomics feature samples; and the plurality of voxel features are screened based on a second screening factor to obtain a plurality of voxel feature samples.

Each of the first screening factor and the second screening factor includes an expression category label of a target gene of the sample object.

In the present embodiment, the plurality of radiomics features and the plurality of voxel features may be respectively screened based on the expression category label of the target gene, the expression category label of the target gene represents an expression category of the target gene of the sample object, wherein the expression category of the target gene includes a mutant type and a wild type, a label of the mutant type is 1, and a label of the wild type is 0.

That the plurality of radiomics features are screened based on the expression category of the target gene may mean that the radiomics features with higher correlation to the expression category of the target gene in the plurality of radiomics features are screened. Similarly, that the plurality of voxel features are screened based on the expression category of the target gene may mean that the voxel features with higher correlation to the expression category of the target gene in the plurality of voxel features are screened.

Exemplarily, if an expression category of a target gene of sample object A is a mutant type, radiomics features with higher correlation to the mutant type in the plurality of radiomics features may be screened, and the voxel features with higher correlation to the mutant type in the plurality of voxel features may be screened. If an expression category of a target gene of sample object B is a wild type, radiomics features with higher correlation to the wild type in the plurality of radiomics features may be screened, and voxel features with higher correlation to the wild type in the plurality of voxel features may be screened. That is to say, for different sample objects, radiomics features and voxel features with higher correlation to expression categories of target genes of the sample objects may be screened according to the expression categories.

In the present embodiment, the correlation between each of the radiomics features and the expression category label may be obtained by calculating a relation value between each of the radiomics features and the expression category label. The correlation between each of the voxel features and the expression category label may be obtained by calculating a relation value between each of the voxel features and the expression category label.

In reality, the first screening factor may further include other screening factors other than the expression category label of the target gene, such as clinical data and a tumor grade. That is to say, for the plurality of radiomics features, screening may be performed once based on each screening factor to obtain radiomics features respectively screened based on each screening factor, and then, the radiomics features respectively screened based on the various screening factors are combined and de-duplicated to obtain the plurality of screened radiomics feature samples.
Step S203: training samples are constructed based on the plurality of radiomics feature samples and the plurality of voxel feature samples.

In the present embodiment, for each sample object, the plurality of corresponding radiomics feature samples and voxel feature samples may be screened, then, the plurality of radiomics feature samples and voxel feature samples screened for each sample object are used as a sample set, and the expression category label of the sample object is used as a label of the sample set to construct a loss function in subsequent model training.

In this way, a plurality of sample objects form a plurality of sample sets, the plurality of sample sets form the training samples, wherein each sample set includes a plurality of radiomics feature samples and voxel feature samples corresponding to one sample object and a corresponding expression category label.
Step S204: a preset model is trained by taking the training samples as inputs to obtain the classification model, wherein the classification model is configured to predict an expression category of the target gene.

In the present embodiment, the plurality of radiomics feature samples and voxel feature samples in each sample set may be inputted to the preset model for training after being fused, wherein fusion may mean that the plurality of radiomics feature samples and voxel feature samples are combined into a feature set, and each feature sample in the feature set is inputted to the preset model.

The preset model may be a classifier, for example, a DenseNet network is used as the classifier, wherein a connection among all layers located in front of each layer of the DenseNet is established on the layer. In this way, an error signal may be easily propagated to earlier layers, and then, the earlier layers may obtain directly supervision from a final classification layer. In this way, the phenomenon of gradient disappearance can be relieved, and model overfitting can be avoided.

After repeatedly training, when the preset model converges, or a loss value approaches to the minimum value, the classification model can be obtained, wherein the classification model can be configured to predict an expression category of a target gene of the tumor patient.

The radiomics features and the voxel features in the present disclosure may be both expressed in a way of feature vectors.

The target gene in the present disclosure may include any one of a TERT gene promoter, a subtype (mutant type/wild type) of an IDH genetype of a gene, a deletion state (deletion/undeletion) of a 1p/19q chromosome, and a methylation state (methylation/unmethylation) of MGMT. It is only necessary to label the expression category label of the target gene in advance. For example, for the TERT gene promoter, its expression category includes a mutant type and a wild type; for the IDH genetype, its expression category includes a mutant type and a wild type; for the 1p/19q chromosome, its expression category includes a deletion category and an undeletion category; and for the MGMT, its expression category includes a methylation category and an unmethylation category.

In an optional example, there may be one or more target genes for training the classification model; under the condition that there is one target gene, the classification model may predict the expression category of one target gene; under the condition that there are more target genes, the classification model may predict expression categories of various genes at the same time, for example, the classification model may output the mutant category of the TERT gene promoter, the deletion state of the 1p/19q chromosome and the methylation state of the MGMT at the same time. In this case, an expression category label may be prepared for each gene, so that the classification model learns the correlation between each of the voxel features and the radiomics features and each of the plurality of target genes at the same time.

By adopting the technical solution in the embodiment of the present disclosure, on one hand, the expression category label may represent the expression category of the target gene, in this way, the radiomics feature samples and the voxel feature samples which are closely correlated to the expression category of the target gene may be screened by taking the expression category of the target gene as a physiological parameter, and then, the preset model is trained by taking the screened radiomics feature samples and voxel feature samples as the training samples, so that the classification model can learn the correlation between each of the morphological features of the tumor region and the expression category of the target gene, the interpretability of the classification model is improved, and then, the accuracy of predicting the expression category of the target gene based on an image of the tumor region is improved.

On the other hand, the training samples not only include the radiomics features of the tumor region, but also include the voxel features of the tumor region, wherein the radiomics features may reflect two-dimensional features such as a slice texture and a shape of the tumor region, and the voxel features may reflect three-dimensional features such as a spatial three-dimensional morphology of the tumor region, so that the richness of the training samples can be improved, and then, the accuracy of the classification model is improved.

### <Extraction of Radiomics Features>

In an optional example, in order to improve the richness and fineness of the extracted radiomics features, two measures are put forward so that the extracted radiomics features can describe the morphological features of the tumor region more finely. Measure A serving as one of the measures is to perform radiomics feature extraction on three subregions of the image of the tumor region, thereby the morphological features of each subregion of the tumor region are reflected for describing morphological features of the different subregions of the tumor region. Measure B serving as the other measure is to perform fine-granularity radiomics feature extraction on the image of the tumor region. Specifically, features for describing different morphological characteristics of a tumor, such as features for describing the fineness of a surface (a slice of MRI) of the tumor and features for describing an appearance shape of the tumor, may be extracted.

Measure A and measure B may be combined, that is, multi-fine-granularity radiomics feature extraction may be performed on an image in each subregion.

Measure A: a first subregion image belonging to a tumor non-enhancement region, a second subregion image belonging to a tumor enhancement region and a third subregion image belonging to a peritumoral edema region are extracted from image samples of the tumor region; and feature extraction is respectively performed on the first subregion image, the second subregion image and the third subregion image to obtain the plurality of radiomics features.

In the present measure A, three-time image segmentation may be performed on the tumor region, and an image of one of the subregions is obtained by each-time image segmentation; and next, feature extraction is performed on the image of each subregion. The subregions include the tumor non-enhancement region, the tumor enhancement region, and the peritumoral edema region; the tumor non-enhancement region refers to an enhanced tumor region in a tumor region, i.e., a nucleus of a tumor; the tumor enhancement region refers to an enhanced region surrounding the nucleus of the tumor and is composed of enhanced tumor voxels; and the peritumoral edema region of the tumor refers to an edema region of the tumor.

In the present embodiment, feature extraction may be performed on the first subregion image to obtain a plurality of first radiomics features belonging to the first subregion image, feature extraction may be performed on the second subregion image to obtain a plurality of second radiomics features belonging to the second subregion image, and feature extraction may be performed on the third subregion image to obtain a plurality of third radiomics features belonging to the third subregion image. Moreover, the plurality of first radiomics features, the plurality of second radiomics features and the plurality of third radiomics features are combined to obtain the plurality of radiomics features of the sample object.

The quantity of the radiomics features extracted for each of the different subregion images may be the same, for example, N radiomics features are extracted from each of the different subregion images. In this way, a total of 3N radiomics features are extracted from the three subregion images.

When measure A is adopted, the tumor region is divided into a tumor core region, an enhanced tumor core region and an overall tumor region according to different expression degrees of tumor cells in the tumor region. Then, when the radiomics features are extracted, it is also possible to perform regional feature extraction on regions where there are different expression degrees of the tumor cells, thereby fine-granularity feature extraction on the tumor region is realized. The extracted radiomics features can fully reflect the morphological features of the tumor region and morphological features of the tumor cells at the different expression degrees, thereby the richness of the training samples is enhanced.

Measure B: wavelet images and LoG images of the image samples of the tumor region are acquired; multi-scale feature extraction is respectively performed on the image samples of the tumor region, the wavelet images and the LoG images to obtain first-order statistics features, texture features and morphological features of the tumor region; and the first-order statistics features, the texture features and the morphological features of the tumor region are combined to obtain the plurality of radiomics features.

In the measure B, the wavelet images may refer to images obtained by performing wavelet transform on the image samples of the tumor region, and the LoG images may refer to edge images of the tumor region, which are finally obtained by solving first-order derivatives of the image samples of the tumor region.

Multi-scale feature extraction may be respectively performed on the image samples of the tumor region, the wavelet images and the LoG images, and each scale corresponds to one dimension, and may specifically include a first-order statistics dimension, a texture dimension and a morphological dimension.

The first-order statistics features, the texture features and the morphological features may be extracted from the image samples of the tumor region, the first-order statistics features and the texture features may be extracted from the wavelet images, and the first-order statistics features and the texture features may be extracted from the LoG images.

The first-order statistics features of the tumor region may be extracted from the first-order statistics dimension. Specifically, the first-order statistics dimension may be a feature value calculated based on pixel gray-level distribution of the image samples, includes morphological features and histogram features, and may reflect the overall morphological features of the tumor region.

The morphological features of the tumor region may be extracted from the morphological dimension. Specifically, the morphological features may be feature values calculated based on contour lines of the tumor region in the image samples, and may reflect a shape and structure of a tumor in the tumor region.

The texture features of the tumor region may be extracted from the texture dimension. Specifically, the texture features in the image samples of the tumor region may be extracted by adopting a statistical method, a geometric method, and a model method, wherein the statistical method may include a gray-level co-occurrence matrix (GLCM) method, a semivariogram, a texture spectrum method, etc. The model method may include a random field model method. The texture features may be configured to describe surface properties of the tumor, such as the thickness and density of the surface, and other features.

During specific implementation, the first-order statistics features and the texture features of the wavelet images may be extracted, the wavelet images are obtained after the image samples are denoised, and the extracted texture features and first-order statistics features include fewer noisy points, thereby contrast with texture features and first-order statistics features extracted from original image samples is formed to obtain multi-dimensional features of the image samples under different scales.

A morphological structure of the tumor region may be outlined based on the LoG images, and thus, different-form feature extraction may be performed on the edge images to obtain the first-order statistics features and the texture features of the LoG images. In this way, morphological features and surface property features of edge lines of the tumor region can be extracted.

In an implementation solution in which measure B is adopted, feature extraction may be respectively performed on the original image samples, the denoised image samples and the edge images of the tumor region in different feature ways, it may be understood that the first-order statistics features and the texture features under different concern points are extracted according to the different concern points, in this way, the first-order statistics features and the texture features under the different concern points may be configured to describe the morphological features of the tumor region under different observation angles, then, the morphological features of the tumor region can be comprehensively reflected, and thus, the richness of the training samples is enhanced.

In reality, measure A and measure B may be combined for use, that is to say, for each subregion image, multi-dimensional feature extraction may be performed on the subregion image to obtain a plurality of radiomics features of each subregion image under each dimension. In this way, not only is feature extraction performed on a fine-granularity region of the tumor region, but also radiomics features are extracted for each fine-granularity region from different observation angles.

In another optional example, the image samples of the tumor region of the sample object may include a T1-weighted image (T1w), a T2-weighted image (T2w), a contrast-enhanced T1-weighted image (T1WCE), and a T2 fluid-attenuated inversion recovery image (T2-FLAIR). During feature extraction, feature extraction may be performed on all of the four types of images.

During specific implementation, the plurality of types of image samples of the tumor region of the target object may be acquired, wherein the plurality of types include a T1-weighted type, a T2-weighted type, a contrast-enhanced T1-weighted type, and a T2 fluid-attenuated inversion recovery type; feature extraction is respectively performed on all the types of image samples; and the radiomics features respectively corresponding to each of the types of extracted image samples are combined to obtain the plurality of radiomics features.

In the present optional example, taking the brain glioma as an example, commonly-used sequences for MRI include T1-weighted (T1), contrast-enhanced T1-weighted (T1c), T2-weighted (T2) and fluid-attenuated inversion recovery (FLAIR) images. Images in different modes are referred to as a mode which may provide complementary information to analyze different glioma subregions. For example, T2 and FLAIR highlight peritumoral edema which is assigned as the overall tumor. T1 and T1c highlight a tumor without peritumoral edema, which is assigned as a tumor core. In T1c, a high-strength enhancement region of the tumor core may also be observed, and is referred to as an enhanced tumor core. Therefore, by applying multi-mode images, the non-determinacy of information can be reduced, and the accuracy of clinical diagnosis and segmentation can be improved.

In this way, for the image sample in each mode, corresponding radiomics features may be extracted. During specific implementation, for the image sample in each mode, the images of the three subregions are extracted from the image samples in the mode (the above-mentioned measure A); next, multi-dimensional feature extraction is respectively performed on the image sample of each subregion (the above-mentioned measure B) to obtain a plurality of radiomics features of the image sample in each mode; and next, the plurality of radiomics features extracted from the image samples in the above-mentioned four modes are combined to obtain the plurality of radiomics features of the sample object.

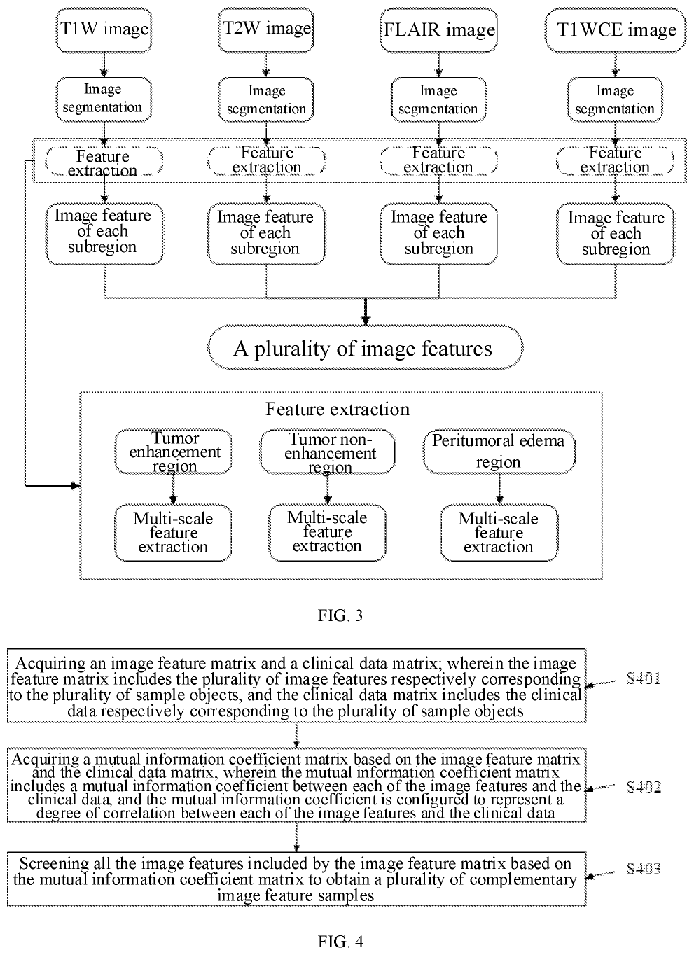

With reference to FIG. 3, a schematic diagram of a process that radiomics features are completely extracted in the present disclosure is shown. As shown in FIG. 3, the image samples (FLAIR images) of a T1-weighted type (a TIW image), a T2-weighted type (a T2W image), a contrast-enhanced T1-weighted type (a T1WCE image) and a T2 fluid-attenuated inversion recovery type are included. It should be noted that the image samples in the four modes are all three-dimensional images.

Image segmentation is performed on the image sample in each mode to obtain a first subregion image, a second subregion image and a third subregion image. The image samples in the different modes are configured to highlight features of different subregions of different tumor regions, and therefore, feature extraction is performed on the image sample of each segmented subregion.

During feature extraction, measure B may be utilized to obtain the first-order statistics features, the morphological features and the texture features of each subregion image, the first-order statistics features and the texture features of the wavelet images as well as the first-order statistics features and the texture features of the LoG images.

During actual feature extraction, reference may be made to the following examples:
for the image sample in each subregion, eighteen first-order statistics features and sixteen morphological features are extracted; for the texture dimension, texture features extracted in different ways may be obtained by using different texture feature extraction ways, and specifically include twenty-four features of a gray-level co-occurrence matrix (GLCM), sixteen features of a gray-level run length matrix (GLRLM), sixteen features of a gray-level size zone matrix (GLSZM), fourteen features of a gray-level dependence matrix (GLDM), and five features of a neighbouring gray-level difference matrix (NGTDM); and
an LoG filter image (sigma: [1.0, 2.0, 3.0, 4.0, 5.0]) has ninety first-order statistics features and three hundred and seventy-five texture features, and a wavelet filter image (LLH, LHL, LHH, HLL, HLH, HHL, HHH, LLL) has one hundred and forty-four first-order statistics features and six hundred texture features.

That is to say, for the image sample of each subregion in each mode, one thousand three hundred and eighteen radiomics features may be obtained. In this way, for the three subregions including the tumor enhancement region, the tumor non-enhancement region, and the peritumoral edema region, a quantity of the radiomics features of the image sample in each mode is three thousand nine hundred and fifty-four which is a product of one thousand three hundred and eighteen and three, and a total quantity of the radiomics features of the image samples in the four modes is fifteen thousand eight hundred and sixteen which is a product of four and three thousand nine hundred and fifty-four.

Of course, above descriptions are only exemplary descriptions. In reality, the quantity of the extracted radiomics features may be determined according to actual demands.

As mentioned above, in the present disclosure, the plurality of radiomics features are screened, which may mean that a plurality of radiomics features are repeatedly screened, each screening may be based on different screening factors. In this way, the repeatedly-screened radiomics features may be combined and de-duplicated to obtain the screened radiomics feature samples. How to screen the radiomics features and the voxel features will be respectively introduced as below.

### <Process for Screening Radiomics Features>

Screening radiomics features may include screening a plurality of radiomics features of single sample object, or screening all radiomics features of all sample objects. During screening a plurality of radiomics features of single sample object, screening may be performed according to an expression category label and a tumor grading label of the tumor region; and during screening all radiomics features of all sample objects, screening may be performed according to clinical data.

During specific implementation, the process that the plurality of radiomics features of the single sample object are screened is described as follows:
in an optional example, the first screening factor may include the expression category label and the tumor grading label of the tumor region; wherein the tumor grading label is configured to label a tumor grade of the sample object, wherein the tumor grade refers to a histological grade of the tumor, and is configured to represent a malignant degree index of the tumor.

It should be noted that the expression category label and the tumor grading label in the present disclosure are both obtained after the sample object is confirmed to suffer from a tumor by diagnosis, that is, they may be an expression category of a target gene and a tumor grade of a patient confirmed to suffer from a tumor by diagnosis.

The radiomics features may be screened respectively based on the expression category label and the tumor grading label, and the radiomics features screened based on the expression category label and the tumor grading label are de-duplicated to obtain the radiomics feature samples.

During specific implementation, the plurality of radiomics features may be screened based on a first relation value between each of the radiomics features and the expression category label to obtain a plurality of first radiomics features; the plurality of radiomics features may be screened based on a second relation value between each of the radiomics features and the tumor grading label to obtain a plurality of second radiomics features; and next, the plurality of first radiomics features and the plurality of second radiomics features are de-duplicated to obtain the plurality of radiomics feature samples.

The first relation value is configured to represent a degree of correlation between each of the radiomics features and mutation of the target gene; and the second relation value is configured to represent a degree of correlation between each of the radiomics features and a tumor grade.

Features significantly correlated to a TERT state label may be selected by using a Mann-Whitney U test method, wherein the Mann-Whitney U test method is configured to evaluate whether the two sampled crowds may be from the same crowd.

Specifically, the sample objects are divided into two groups x1 and x2 according to TERT state category labels 0 and 1, wherein an expression category label of TERT of the sample objects in x1 is 0, and an expression category label of TERT of the sample objects in x2 is 1; and next, the Mann-Whitney U test between the samples x1 and x2 is calculated to obtain a p-value, i.e., the first relation value of each radiomics feature of each sample object, the first relation value may reflect a degree of correlation between each of the radiomics features of the sample object and a state label, then, the radiomics features with p-value<0.05 are retained, and thus, the first screening for the radiomics features is completed.

It is also possible to select features significantly correlated to the tumor grade (high-grade glioma/low-grade glioma) label by using a Mann-Whitney U test method, wherein the tumor grading label may include 0 and 1, 0 represents a high-grade tumor, 1 represents a low-grade tumor, the sample objects are divided into two groups x3 and x4 according to the tumor grading labels 0 and 1, wherein the tumor grading label of the sample objects in x3 is 0, the tumor grading label of the sample objects in x4 is 1; and next, the Mann-Whitney U test between the samples x3 and x4 is calculated to obtain a p-value, i.e., the second relation value of each radiomics feature of each sample object, the second relation value may reflect a degree of correlation between each of the radiomics features of the sample object and the tumor grading label, then, the radiomics features with p-value<0.05 are retained, and thus, the second screening for the radiomics features is completed.

Next, after the plurality of first radiomics features and the plurality of second radiomics features are combined, repeated radiomics features are removed to obtain the plurality of radiomics feature samples.

It should be noted that the first screening (screening based on the expression category label of the target gene) and the second screening (screening based on the tumor grading label) are independent from each other.

In yet another optional example, radiomics features with higher distinguishing capacities may be selected firstly by adopting a variance selection method, and next, a plurality of radiomics feature samples are screened from the radiomics features with higher distinguishing capacities based on the first screening factor.

During specific implementation, a variance corresponding to each of the radiomics features may be determined, and radiomics features of which variances are greater than a second variance threshold are retained to obtain a plurality of candidate radiomics features. Then, the plurality of candidate radiomics features are screened based on the first screening factor to obtain the plurality of radiomics feature samples.

By using the variance selection method, features useful for distinguishing the samples may be selected, that is to say, radiomics features with stronger feature expression may be selected. Specifically, if a variance of a radiomics feature approaches to 0, the sample object is represented to basically have no differences on the radiomics feature, and the radiomics feature is not useful for the distinguishing the sample objects.

Specifically, a threshold may be set, radiomics features of which variances are greater than the threshold are retained to obtain the radiomics features selected by using the variance selection method. In reality, data standardization may be performed, by using z-score, on the radiomics features selected by using the variance selection method, and next, the data-standardized radiomics features are screened based on the first screening factor to obtain the radiomics feature samples.

When such an implementation is adopted, radiomics features incapable of greatly distinguishing the samples in the extracted radiomics features may be removed firstly, and then, the retained radiomics features have strong feature expression, so that the feature expression strength of the radiomics features screened subsequently is improved, a quantity of calculation for subsequent feature screening is also reduced, and the feature screening efficiency is increased.

The process that the plurality of radiomics features of all the sample objects are screened is described as follows:
during specific implementation, all the radiomics features are screened based on a third screening factor to obtain complementary radiomics feature samples; wherein the third screening factor includes clinical data respectively corresponding to the plurality of sample objects.

Correspondingly, the training samples may be constructed based on the plurality of radiomics feature samples, the plurality of voxel feature samples, and the plurality of complementary radiomics feature samples.

Specifically, as mentioned above, the training samples include a plurality of sample sets, and each sample set includes a plurality of radiomics feature samples and a plurality of voxel feature samples which correspond to one sample object, and the plurality of complementary radiomics feature samples of the sample object.

In this way, the radiomics feature samples screened from the plurality of sample objects form a radiomics feature subset 1, the plurality of complementary radiomics feature samples form a radiomics feature subset 2, the voxel feature samples form a voxel feature subset, and the radiomics feature subset 1, the radiomics feature subset 2 and the voxel feature subset are used as the training samples for training the preset model.

During specific implementation, with reference to FIG. 4, a schematic flow diagram of steps for screening radiomics features based on clinical data is shown. As shown in FIG. 4, the method may specifically include the following steps:
step S401: a radiomics feature matrix and a clinical data matrix are acquired; wherein the radiomics feature matrix includes the plurality of radiomics features respectively corresponding to the plurality of sample objects, and the clinical data matrix includes the clinical data respectively corresponding to the plurality of sample objects;
step S402: a mutual information coefficient matrix is acquired based on the radiomics feature matrix and the clinical data matrix, wherein the mutual information coefficient matrix includes a mutual information coefficient between each of the radiomics features and the clinical data, and the mutual information coefficient is configured to represent a degree of correlation between each of the radiomics features and the clinical data; and
step S403: all the radiomics features included in the radiomics feature matrix are screened based on the mutual information coefficient matrix to obtain a plurality of complementary radiomics feature samples.

In the present embodiment, the radiomics features of the plurality of sample objects may be screened based on the respectively corresponding clinical data to obtain the complementary radiomics feature samples screened from each sample object. During specific implementation, the correlation between each radiomics feature and the clinical data may be measured by means of mutual information. Specifically, if there are N sample objects, the radiomics feature matrix is set as A_{N*M}, and the clinical data matrix is set as B_{N*S}.

M is a quantity of the radiomics features extracted from each sample object. For example, in above example, fifteen thousand eight hundred and sixteen features are extracted, and thus, M is fifteen thousand eight hundred and sixteen. Of course, after parts of radiomics features are selected by using the variance selection method, M is a quantity of the radiomics features selected by using the variance selection method. S is a quantity of the clinical data of each sample object.

In the present disclosure, the clinical data includes age, gender, systolic blood pressure, diastolic blood pressure, disease history, malignant tumor history, medication information, surgical condition, survival time, etc. Examples are shown as the following table:

| | | | Systolic | Diastolic | | Malignant | | | Survival |
|---|---|---|---|---|---|---|---|---|---|
| Reference | | | blood | blood | Disease | tumor | Medication | Surgical | time |
| numeral | Age | Gender | pressure | pressure | history | history | information | condition | (month) |
| 1 | 31 | Female | 108 mmHg | 70 mmHg | Diabetes | No | Temozolomide | GTR | 4.69 |
| 2 | 67 | Male | 130 mmHg | 80 mmHg | Hypertension | No | No | STR | 7.68 |
| | | | | | and cerebro | | | | |
| | | | | | vascular | | | | |
| | | | | | diseases | | | | |

All data in the clinical data may be converted into clinical data features, and S represents a quantity of the clinical data features. The process that the clinical data is converted into the clinical data features may be described as follows:

The numeric clinical data is normalized, for example, the age, the systolic blood pressure and the diastolic blood pressure are normalized. Character-string-type clinical data is firstly converted into numeric information, for example, data such as the gender, the disease history, the malignant tumor history, the medication information and the surgical condition is quantized. For example, for the gender, male is represented by 1, and female is represented by 0; for the disease history, diabetes is represented by 1, hypertension is represented by 2, cerebrovascular diseases are represented by 3, and next, they are converted into vectors for representation; wherein feature discretization is performed on clinical data for the survival time, and the clinical data is divided into three categories based on a standard that zero to three years are represented by 1, three to five years are represented by 2, and five or more years are represented by 3.

Of course, in reality, it is also possible to perform one-hot encoding on each clinical data to obtain the clinical data feature corresponding to each clinical data.

The mutual information coefficient matrix may be acquired based on the radiomics feature matrix and the clinical data matrix. Specifically, mutual information coefficients between each radiomics feature of each sample object and a different clinical data feature of the sample object may be calculated to obtain the mutual information coefficient matrix C_{S*M}. In this way, each row of the coefficient matrix C_{S*M} represents the mutual information coefficients respectively corresponding to the M radiomics features of one sample object, next, K best radiomics features may be selected from each row, and features selected from S rows (S clinical data features) are combined and de-duplicated to obtain the plurality of complementary radiomics feature samples.

The plurality of complementary radiomics feature samples may be grouped according to the sample objects to which the complementary radiomics feature samples respectively belong to obtain the complementary radiomics feature sample corresponding to each sample object, and then, the complementary radiomics feature sample corresponding to each sample object may be divided into the sample set to which the sample object belongs so as to be used as a training sample.

By using the mutual information coefficients, the correlation between each of the radiomics features and each of the clinical data features may be measured, and therefore, radiomics features, correlated to the clinical data features, of each sample object may be screened, that is to say, radiomics features closely correlated to a state of an illness of a patient may be screened based on the clinical data to train a model, so that the interpretability of the model is improved.

In another aspect, the mutual information coefficient matrix is acquired based on the radiomics feature matrix and the clinical data matrix, in this way, the corresponding complementary radiomics feature samples may be screened by means of the mutual information coefficient matrix. Therefore, compared with single calculation of the correlation between each of the radiomics features and each of the clinical data features of each sample object, by using this method, the complementary radiomics feature samples respectively corresponding to the plurality of sample objects can be screened at one time, thereby the screening efficiency increasing. Moreover, the radiomics features and the clinical data features of the different sample objects are brought into a unified matrix space for calculation. Therefore, when the complementary radiomics feature samples of one sample object are screened, a medical correlation between each of the clinical data features and each of the radiomics features is constructed based on the plurality of sample objects by means of the correlation between each of the clinical data features and each of the radiomics features of other sample objects, so that the accuracy of the screened complementary radiomics feature samples is improved, that is, the radiomics feature samples capable of truly reflecting the clinical data are screened.

### <Process for Screening Voxel Features>

In an optional example, the screening factor for screening the voxel features may include an expression category label. Of course, during screening, the voxel features may be primarily screened firstly to obtain voxel features with strong feature expression, and then, the voxel features with strong feature expression are screened based on the expression category label, so that a quantity of the voxel features calculated by the classification model and a quantity for calculation caused when the voxel feature are screened are reduced.

During specific implementation, a variance of each of the voxel features may be acquired, and voxel features of which variances are greater than a first variance threshold are retained to obtain a plurality of candidate voxel features; and taking the expression category label as a predication label and the plurality of candidate voxel features as inputs, the plurality of voxel feature samples are screened from the plurality of candidate voxel features by using a linear regression model.

In the present example, the variance of each of the voxel features may be still calculated, and next, the voxel features of which the variances are greater than the first variance threshold are retained, wherein the first variance threshold may be different from the above-mentioned second variance threshold. The linear regression model may be a least absolute shrinkage and selection operator (LASSO) regression model. Specifically, for the retained candidate voxel features, voxel feature selection may be performed by adopting an LASSO regression L1 regularization algorithm. Specifically, taking the plurality of candidate voxel features as input features of an LASSO, a predication label of the LASSO is the expression category label of the target gene, and thus, a set of voxel feature samples selected by the LASSO is obtained.

In reality, there is a certain correlation between a tumorigenesis position and the expression category of the target gene. Therefore, as shown in FIG. 1, in an optional example, a position of the tumor region on a human body to which the tumor region belongs may be further determined, in this way, position information of the position may be used as complementary features of the training samples to train the classification model.

Specifically, position information corresponding to a glioma region is determined based on an image sample of the glioma region; and position features corresponding to the position information are acquired; wherein the position information includes a brain region to which the glioma region belongs, and/or position coordinates of the glioma region in the brain.

Correspondingly, the training samples may be constructed based on the position features, the plurality of radiomics feature samples and the plurality of voxel feature samples.

In the present implementation, for the brain glioma, there is a certain correlation between a region where glioma occurs and the expression category of the target gene. Therefore, in order to describe such a correlation, the position of the glioma region in the brain may be acquired, that is, the position information corresponding to the glioma region is acquired.

The position of the glioma region may be determined according to a magnetic resonance image of the brain, and then, the position information is determined based on the brain region to which the position belongs. The position information may include the brain region to which the glioma region belongs or the position coordinates of the glioma region in the brain, or not only include the brain region to which the glioma region belongs, but also include the position coordinates of the glioma region in the brain. Specifically, the position coordinates may be center coordinates of the glioma in the brain, i.e., a spatial position of the glioma in the brain. In an example, the brain region may include a cerebrum, a cerebellum and a brainstem.

In another example, the brain is divided in detail into one hundred and sixteen regions of interest (ROI) according to an Anatomical Automatic Labeling (AAL) atlas which is a digital atlas of a brain structure and is generally configured to position an activity region of the brain in functional neuroimaging studies, and therefore, the brain region may include the one hundred and sixteen ROIs.

When the position information is converted into the position features, the position coordinates may be represented by numerical values. The brain region to which the glioma region belongs may be represented by a label indicating whether the glioma region belongs to each of the above-mentioned brain regions. Taking that the brain region includes the cerebrum, the cerebellum and the brainstem as an example, if a tumor belongs to the region, the tumor is expressed as 1; if the tumor does not belong to the region, the tumor is expressed as 0; and if glioma is distributed on the cerebellum and the brainstem, the position features are expressed as [0, 1, 1].

By adopting the technical solution in the present implementation, position features of a region to which a tumor belongs may be fused to provide a reference of a tumor position for predicting the expression category of the target gene, and the expression category of the target gene may be relatively accurately predicted based on the correlation between the tumorigenesis position and the expression category of the target gene.

The process that the preset model is trained by using the training samples to obtain the classification model may be described as follows:
the training samples are inputted to the classification model to obtain a predicted expression category, outputted by the classification model, of the target gene; a loss value of the preset model is determined based on the predicted expression category and the expression category label; parameters of the preset model are updated based on the loss value; and next, a preset model satisfying a training ending condition is taken as the classification model, wherein the training ending condition is that the classification model converges or reaches a time quantity of preset updating.

In the present implementation, as mentioned above, the sample set of each sample object, i.e., the radiomics feature samples, the voxel feature samples and the complementary radiomics feature samples screened from each sample object, may be inputted to the preset model. The preset model is processed with different scales based on the radiomics feature samples, the voxel feature samples and the complementary radiomics feature samples, and thus, the expression category, i.e., the predicted expression category, of the target gene of the sample object is predicted. Then, a loss function is constructed based on the expression category label and the predicted expression category of the sample object, the loss value of the preset module is calculated, the parameters of the preset model are continuously updated based on the loss value, and finally, the classification model is obtained.

In the training ending condition, the condition that the classification model converges may mean that the loss value is smaller than or equal to a preset loss value, or the loss value is no longer smaller. The time quantity of preset updating may be set according to actual demands.

By adopting the method for acquiring the classification model in the present disclosure, the training samples are the radiomics feature samples and voxel feature samples screened based on the expression category label of the target gene and having stronger correlation to the expression category of the target gene, therefore, the classification model learns the correlation between each of the radiomics features and the voxel features and the expression category of the target gene, and then, the interpretability of the classification model is improved.

After the above-mentioned classification model is obtained by training, the training samples inputted to the preset model are valid radiomics feature samples and voxel feature samples screened based on the clinical data and the expression category of the target gene, in this way, the preset model may learn the correlation between each of these features correlated to the mutation of the target gene and the expression category of the target gene in the training process, that is to say, the medical interpretability of the classification model can be improved, then, the classification model can predict the expression category of the target gene based on the radiomics features and the voxel features.

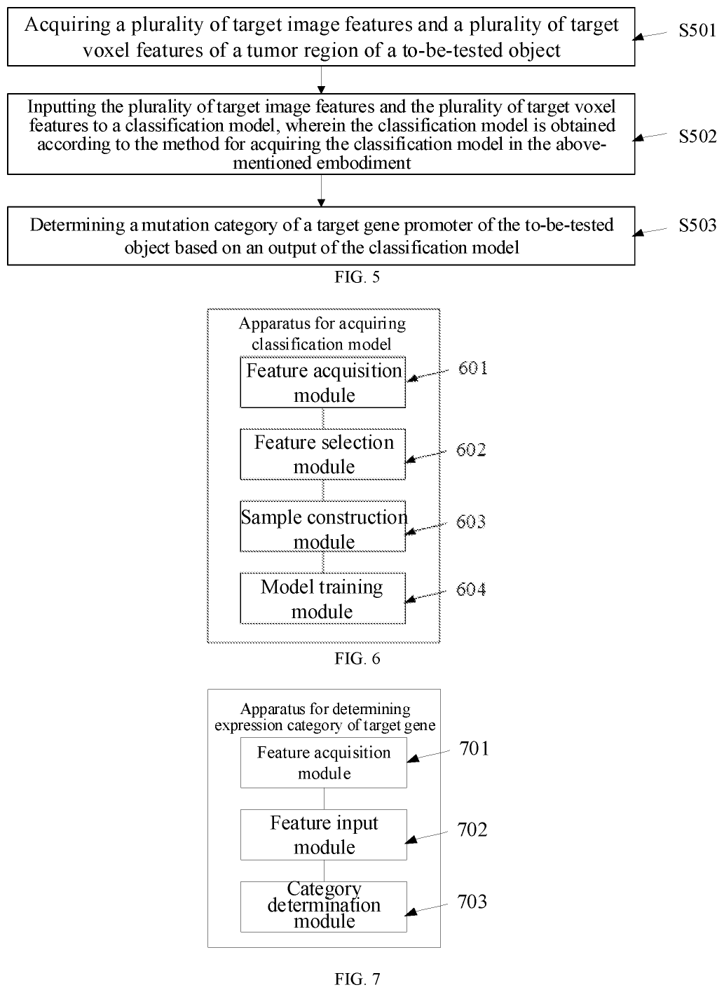

In this way, in another embodiment, a method for determining an expression category of a target gene is provided in the present disclosure. With reference to FIG. 5, a flow diagram of steps of a method for determining an expression category of a target gene is shown. As shown in FIG. 5, the method includes the following steps:
step S501: a plurality of target radiomics features and a plurality of target voxel features of a tumor region of a to-be-tested object are acquired;
step S502: the plurality of target radiomics features and the plurality of target voxel features are inputted to a classification model, wherein the classification model is obtained according to the method for acquiring the classification model in the above-mentioned embodiment; and
step S503: an expression category of a target gene of the to-be-tested object is determined based on an output of the classification model.

In the present embodiment, the to-be-tested object may indicate a patient belonging to the expression category of the to-be-tested target gene, wherein a magnetic resonance image of the tumor region of the to-be-tested object may be acquired. Specifically, the magnetic resonance image may include the images in the four modes in the above-mentioned embodiment, i.e., an image of a T1-weighted type, an image of a T2-weighted type, an image of a contrast-enhanced T1-weighted type, and an image of a T2 fluid-attenuated inversion recovery type. Next, images of three subregions may be segmented from the image in each mode, multi-scale feature extraction is respectively performed on the image of each subregion, and the first-order statistics features, the texture features and the morphological features in the above-mentioned embodiment may be acquired by the multi-scale feature extraction, and thus, the plurality of target radiomics features corresponding to the to-be-tested object are obtained.

The process that the target voxel features of the to-be-tested object are acquired may be referred to the description for the process that the voxel features of the sample object in the above-mentioned implementation, and will be no longer repeated herein.

Next, the plurality of target radiomics features and the plurality of target voxel features may be inputted to the classification model. The classification model has learned the correlation between each of the radiomics features and the voxel features and the expression category of the target gene in the acquisition process in the above-mentioned embodiment so as to be capable of predicting the expression category of the target gene based on the radiomics features and the voxel features.

The output of the classification model is a probability that the target gene belongs to each expression category, i.e., a probability that the target gene belongs to a mutant type and a probability that the target gene belongs to a wild type. In reality, a category of which the probability is greater than a preset probability value may be used as the expression category of the target gene.

By adopting the method for determining the expression category of the target gene in the present implementation solution, since the preset model learns the correlation between each of the these features correlated to the mutation of the target gene and the expression category of the target gene in the training process, that is to say, the medical interpretability of the classification model can be improved, then, the classification model can predict the expression category of the target gene based on the radiomics features and the voxel features, and then directly input the target radiomics features and the target voxel features of the to-be-tested object to the classification model in an actual application to obtain the accurate expression category of the target gene without determining a mutation state by using an invasive detection method such as pyrosequencing or polymerase chain reaction (PCR) for the to-be-tested object, the pain of the patient can be greatly relieved.

In an optional example, when the expression category of the target gene of the to-be-tested object is predicted by using the classification model, the radiomics features can also be screened. On one hand, the target radiomics features and the target voxel features with stronger feature expression can be screened; and on the other hand, the target radiomics features having stronger correlation to a tumor grade and clinical data of the to-be-tested object can be screened.

During specific implementation, the process that the target radiomics features with stronger feature expression are screened is described as follows: a variance corresponding to each of the target radiomics features may be determined, and target radiomics features of which variances are greater than a second variance threshold are retained; and a variance corresponding to each of the voxel features is determined, and target voxel features of which variances are greater than a first variance threshold are retained. Correspondingly, the retained target radiomics features and the retained target voxel features may be inputted to the classification model.

The first variance threshold may be the same as the first variance threshold in the above-mentioned embodiment, and the second variance threshold may be the same as the second variance threshold in the above-mentioned embodiment. That is to say, for the to-be-tested object, the target voxel features and the target radiomics features with stronger expression capacities can also be selected by adopting a variance selection method.

During specific implementation, the process that the target radiomics features having stronger correlation to the tumor grade and the clinical data of the to-be-tested object are screened may be described as follows:
a fourth screening factor corresponding to the to-be-tested object is acquired, and the plurality of target radiomics features are screened based on the fourth screening factor; wherein the fourth screening factor includes clinical data and/or tumor grading data of the to-be-tested object.

The fourth screening factor may include the clinical data or the tumor grading data, or include both of the clinical data and the tumor grading data.

Under the condition that only the clinical data is included, a mutual information coefficient between each of the target radiomics features and the clinical data may be calculated, and then, the target radiomics features inputted to the classification model are screened based on the mutual information coefficient; and under the condition that only the tumor grade is included, a third relation value between each of the target radiomics features and the tumor grade may be calculated, and then, the target radiomics features inputted to the classification model are screened based on the third relation value.

Under the condition that the clinical data and the tumor grade are included, the third relation value between each of the target radiomics features and the tumor grading label and the mutual information coefficient between each of the target radiomics features and the clinical data may be determined; and
next, the plurality of target radiomics features are respectively screened based on the third relation value, and the plurality of target radiomics features are screened based on the mutual information coefficient; and then, the target radiomics features screened based on the third relation value and the target radiomics features screened based on the mutual information coefficient are de-duplicated to obtain the screened target radiomics features.

The process that the third relation value and the mutual information coefficient are calculated may be referred to the description in the above-mentioned embodiment, and will be no longer repeated herein.

By adopting such an implementation, the target radiomics features closely correlated to the clinical data of the to-be-tested object and the target radiomics features closely correlated to the tumor grade of the to-be-tested object are screened from the plurality of target radiomics features, and therefore, under the condition that the classification model has the capacity of determining the correlation between each of the radiomics features and the voxel features and the expression category of the target gene, the radiomics features inputted to the classification model are also the target radiomics features closely correlated to the clinical data and the tumor grade of the to-be-tested object. Therefore, the accuracy of predicting the expression category of the target gene can be further improved.

Based on the same inventive concept, an apparatus for acquiring a classification model is further provided by the present disclosure. With reference to FIG. 6, a schematic diagram of a structural framework of an apparatus for acquiring a classification model in the present disclosure is shown. As shown in FIG. 6, the apparatus may specifically include the following modules:
a feature acquisition module 601 configured to, for a tumor region of a sample object, acquire a plurality of radiomics features and a plurality of voxel features of the tumor region;
a feature selection module 602 configured to screen the plurality of radiomics features based on a first screening factor to obtain a plurality of radiomics feature samples; and screen the plurality of voxel features based on a second screening factor to obtain a plurality of voxel feature samples; wherein each of the first screening factor and the second screening factor includes an expression category label of a TERT gene of the sample object;
a sample construction module 603 configured to construct training samples based on the plurality of radiomics feature samples and the plurality of voxel feature samples; and
a model training module 604 configured to train a preset model by taking the training samples as inputs to obtain a classification model, wherein the classification model is configured to predict a mutation category of the target gene.

Optionally, the feature acquisition module 601 includes:
an image segmentation unit configured to extract a first subregion image belonging to a
tumor non-enhancement region, a second subregion image belonging to a tumor enhancement region and a third subregion image belonging to a peritumoral edema region from image samples of the tumor region; and
a feature extraction unit configured to respectively perform feature extraction on the first subregion image, the second subregion image and the third subregion image to obtain the plurality of radiomics features.

Optionally, the feature acquisition module 601 includes:
a multi-type image acquisition unit configured to acquire a plurality of types of image samples of the tumor region, wherein the plurality of types include a T1-weighted type, a T2-weighted type, a contrast-enhanced T1-weighted type, and a T2 fluid-attenuated inversion recovery type;
a feature extraction unit configured to perform feature extraction on each of the plurality of types of image samples, respectively; and
a feature combination unit configured to combine the radiomics features respectively corresponding to each of the plurality of types of extracted image samples to obtain the plurality of radiomics features.

Optionally, the tumor region is a glioma region in a brain, and the apparatus further includes:
a position information acquisition module configured to determine position information corresponding to the glioma region based on an image sample of the glioma region; and
a position feature acquisition module configured to acquire position features corresponding to the position information; wherein the position information includes a brain region to which the glioma region belongs, and/or position coordinates of the glioma region in the brain; and
the sample construction module 603 specifically configured to construct the training samples based on the position features, the plurality of radiomics feature samples and the plurality of voxel feature samples.

Optionally, the first screening factor includes the expression category label and a tumor grading label of the tumor region; and the feature selection module 602 includes a radiomics feature screening unit including:
a first screening subunit configured to screen the plurality of radiomics features based on a first relation value between each of the plurality of radiomics features and the expression category label to obtain a plurality of first radiomics features; wherein the first relation value is configured to represent a degree of correlation between each of the plurality of radiomics features and mutation of the target gene;
a second screening subunit configured to screen the plurality of radiomics features based on a second relation value between each of the plurality of radiomics features and the tumor grading label to obtain a plurality of second radiomics features; wherein the second relation value is configured to represent a degree of correlation between each of the plurality of radiomics features and a tumor grade; and
a de-duplication unit configured to de-duplicate the plurality of first radiomics features and the plurality of second radiomics features to obtain the plurality of radiomics feature samples.

Optionally, a plurality of sample objects are included, and the apparatus further includes:
a radiomics feature rescreening module configured to, for all the plurality of radiomics features included by all the sample objects, screen all the plurality of radiomics features based on a third screening factor to obtain complementary radiomics feature samples; wherein the third screening factor includes clinical data respectively corresponding to the plurality of sample objects; and
the sample construction module 603 specifically configured to construct the training samples based on the plurality of radiomics feature samples, the plurality of voxel feature samples, and the plurality of complementary radiomics feature samples.

Optionally, the radiomics feature rescreening module includes:
a matrix creation unit configured to acquire a radiomics feature matrix and a clinical data matrix; wherein the radiomics feature matrix includes the plurality of radiomics features respectively corresponding to the plurality of sample objects, and the clinical data matrix includes the clinical data respectively corresponding to the plurality of sample objects;
a mutual information coefficient determination unit configured to acquire a mutual information coefficient matrix based on the radiomics feature matrix and the clinical data matrix, wherein the mutual information coefficient matrix includes a mutual information coefficient between each of the plurality of radiomics features and the clinical data, and the mutual information coefficient is configured to represent a degree of correlation between each of the plurality of radiomics features and the clinical data; and
a complementary screening unit configured to screen, based on the mutual information coefficient matrix, all the plurality of radiomics features included by the radiomics feature matrix to obtain a plurality of complementary radiomics feature samples.

Optionally, the second screening factor includes the expression category label, and the feature selection module 602 includes a voxel feature screening unit including:
a first variance determination unit configured to acquire a variance of each of the plurality of the voxel features, and retain voxel features of which variances are greater than a first variance threshold to obtain a plurality of candidate voxel features; and
a voxel screening unit configured to, taking the expression category label as a predication label and taking the plurality of candidate voxel features as inputs, screen the plurality of voxel feature samples from the plurality of candidate voxel features by using a linear regression model.

Optionally, the apparatus further includes:
a second variance determination unit configured to determine a variance corresponding to each of the plurality of the radiomics features, and retain radiomics features of which variances are greater than a second variance threshold to obtain a plurality of candidate radiomics features; and
a radiomics feature screening unit configured to screen the plurality of candidate radiomics features based on the first screening factor to obtain the plurality of radiomics feature samples.

Optionally, the feature acquisition module includes a radiomics feature extraction unit specifically including:
a multi-multidimensional feature extraction subunit configured to acquire wavelet images and LoG images of the image samples of the tumor region; and perform multi-scale feature extraction on the image samples of the tumor region, the wavelet images and the LoG images, respectively, to obtain first-order statistics features, texture features and morphological features of the tumor region; and
a multidimensional feature combination subunit configured to combine the first-order statistics features, the texture features and the morphological features of the tumor region to obtain the plurality of radiomics features.

Optionally, the model training module includes:
an input unit configured to input the training samples to the classification model to obtain a predicted expression category, outputted by the classification model, of a target gene;
a loss determination unit configured to determine a loss value of the classification model based on the predicted expression category and the expression category label;
a parameter updating unit configured to update parameters of the classification model based on the loss value; and
a classification model acquisition unit configured to take a classification model satisfying a training ending condition as the classification model, wherein the training ending condition is that the classification model converges or reaches a time quantity of preset updating.

Based on the same inventive concept, an apparatus for determining an expression category of a target gene is further provided by the present disclosure. With reference to FIG. 7, a schematic diagram of a framework of an apparatus for determining an expression category of a target gene is shown. As shown in FIG. 7, the apparatus may specifically include the following modules:
a feature acquisition module 701 configured to acquire a plurality of target radiomics features and a plurality of target voxel features of a tumor region of a to-be-tested object;
a feature input module 702 configured to input the plurality of target radiomics features and the plurality of target voxel features to the classification model, wherein the classification model is obtained according to the method for acquiring the classification model in the above-mentioned embodiment; and
a category determination module 703 configured to determine an expression category of a target gene of the to-be-tested object based on an output of the classification model.

Optionally, the apparatus further includes:
a first radiomics feature screening module configured to determine a variance corresponding to each of the plurality of target radiomics features, and retain target radiomics features of which variances are greater than a second variance threshold;
a voxel feature screening module configured to determine a variance of each of the plurality of voxel features, and retain target voxel features of which variances are greater than a first variance threshold; and
the feature input module 702 specifically configured to input the retained target radiomics features and target voxel features to the classification model.

Optionally, the apparatus further includes:
a screening factor acquisition module configured to acquire a fourth screening factor corresponding to the to-be-tested object, wherein the fourth screening factor includes clinical data and/or tumor grading data of the to-be-tested object;
a second radiomics feature screening module configured to screen the plurality of target radiomics features based on the fourth screening factor; and
the feature input module 702 specifically configured to input the plurality of screened target radiomics features and the plurality of screened target voxel features to the classification model.

Optionally, the fourth screening factor includes the clinical data and the tumor grading data; and the second radiomics feature screening module includes:
a numerical value determination unit configured to determine a third relation value between each of the target radiomics features and a tumor grading label and a mutual information coefficient between each of the plurality of target radiomics features and the clinical data;
a screening unit configured to screen the plurality of target radiomics features based on the third relation value, and screen the plurality of target radiomics features based on the mutual information coefficient; and
a de-duplication unit configured to de-duplicate the target radiomics features screened based on the third relation value and the target radiomics features screened based on the mutual information coefficient to obtain the screened target radiomics features.

Based on the same inventive concept, an electronic device is further provided by the present disclosure, including a memory, a processor, and a computer program stored in the memory and capable of running on the processor, wherein the processor, when being executed, implements the method for acquiring the classification model, or when being executed, implements the method for determining the expression category of the target gene.

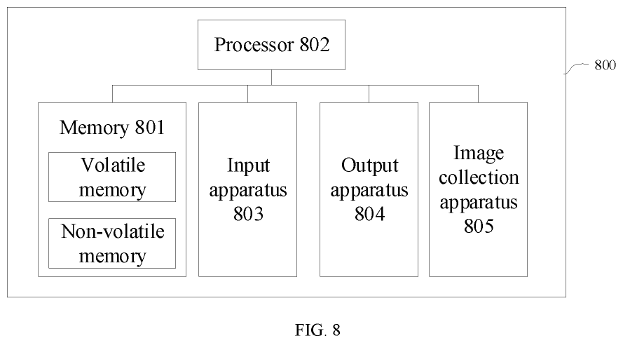

With reference to FIG. 8, a structural block diagram of an electronic device 800 in an embodiment of the present disclosure is shown. As shown in FIG. 8, an electronic device is provided in an embodiment of the present disclosure. The electronic device 800 may be configured to implement the method for acquiring the classification model or the method for determining the expression category of the target gene.

The electronic device 800 may include a memory 801, a processor 802, and a computer program stored in the memory and capable of running on the processor, wherein the processor 802 is configured to implement an image processing method.

As shown in FIG. 8, in an embodiment, the overall electronic device 800 may include an input apparatus 803, an output apparatus 804, and an image collection apparatus 805, wherein when the image processing method in the embodiment of the present disclosure is implemented, the image collection apparatus 805 may acquire an image of a tumor region (including image samples and an image of a tumor region of a to-be-tested object), next, the input apparatus 803 may obtain the image acquired by the image collection apparatus 805, the image may be processed by the processor 802, and the processing may specifically include that: radiomics features and voxel features are extracted, the radiomics features and the voxel features are screened, training samples are constructed based on the screened features to train a preset model, and the output apparatus 804 may output a classification model, or an expression category result outputted by the classification model.

Of course, in an embodiment, the memory 801 may include a volatile memory and a nonvolatile memory, wherein the volatile memory may be understood as a random access memory, and is configured to store and save data. The nonvolatile memory refers to a computer memory in which stored data cannot disappear after a current is turned off. Of course, the computer program for the method for acquiring the classification model or the method for determining the expression category of the target gene in the present disclosure may be stored in a volatile memory and a nonvolatile memory or any one of the both.

Based on the same inventive concept, a computer-readable storage medium is further provided by the present disclosure, wherein a computer program stored in the computer-readable storage medium enables the processor, when being executed, to implement the method for acquiring the classification model, or when being executed, implement the method for determining the expression category of the target gene.

Based on the same inventive concept, a computer program product is further provided by the present disclosure, including a computer program/instruction, wherein the computer program/instruction, when being executed, implements the method for acquiring the classification model, or when being executed, implements the method for determining the expression category of the target gene.

Finally, it should also be noted that, in the present text, relation terms such as first and second are merely intended to distinguish one entity or operation from another entity or operation, and that does not necessarily require or imply that those entities or operations have therebetween any such actual relation or order. Furthermore, the terms “include”, “include” or any variants thereof are intended to cover non-exclusive inclusions, so that processes, methods, articles or devices that include a series of elements do not only include those elements, but also include other elements that are not explicitly listed, or include the elements that are inherent to such processes, methods, articles or devices. Unless further limitation is set forth, an element defined by the wording “including a . . . ” does not exclude additional same element in the process, method, article or device including the element.

The task dispatching method and apparatus, the computing and processing device, the computer program and the computer-readable medium according to the present disclosure have been described in detail above. The principle and the embodiments of the present disclosure are described herein with reference to the particular examples, and the description of the above embodiments is merely intended to facilitate to understand the method according to the present disclosure and its core concept. Moreover, for a person skilled in the art, according to the concept of the present disclosure, the particular embodiments and the range of application may be varied. In conclusion, the contents of the description should not be understood as limiting the present disclosure.

A person skilled in the art, after considering the description and implementing the invention disclosed herein, will readily envisage other embodiments of the present disclosure. The present disclosure aims at encompassing any variations, uses or adaptive alternations of the present disclosure, wherein those variations, uses or adaptive alternations follow the general principle of the present disclosure and include common knowledge or common technical means in the art that are not disclosed by the present disclosure. The description and the embodiments are merely deemed as exemplary, and the true scope and spirit of the present disclosure are presented by the following claims.

It should be understood that the present disclosure is not limited to the accurate structure that has been described above and shown in the drawings, and may have various modifications and variations without departing from its scope. The scope of the present disclosure is merely limited by the appended claims.

The “one embodiment”, “an embodiment” or “one or more embodiments” as used herein means that particular features, structures or characteristics described with reference to an embodiment are included in at least one embodiment of the present disclosure. Moreover, it should be noted that here an example using the wording “in an embodiment” does not necessarily refer to the same one embodiment.

The description provided herein describes many concrete details. However, it may be understood that the embodiments of the present disclosure may be implemented without those concrete details. In some of the embodiments, well-known processes, structures and techniques are not described in detail, so as not to affect the understanding of the description.

In the claims, any reference signs between parentheses should not be construed as limiting the claims. The word “include” does not exclude elements or steps that are not listed in the claims. The word “a” or “an” preceding an element does not exclude the existing of a plurality of such elements. The present disclosure may be implemented by means of hardware including several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are merely intended to explain the technical solutions of the present disclosure, and not to limit them. Although the present disclosure is explained in detail with reference to the above embodiments, a person skilled in the art should understand that he may still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present disclosure.

1. A method for acquiring a classification model, comprising:
for a tumor region of a sample object, acquiring a plurality of radiomics features and a plurality of voxel features of the tumor region;
screening the plurality of radiomics features based on a first screening factor to obtain a plurality of radiomics feature samples; and screening the plurality of voxel features based on a second screening factor to obtain a plurality of voxel feature samples; wherein each of the first screening factor and the second screening factor comprises an expression category label of a target gene of the sample object;
constructing training samples based on the plurality of radiomics feature samples and the plurality of voxel feature samples; and
training a preset model by taking the training samples as inputs to obtain the classification model, wherein the classification model is configured to predict an expression category of the target gene.

2. The method according to claim 1, wherein acquiring the plurality of radiomics features of the tumor region comprises:
extracting a first subregion image belonging to a tumor non-enhancement region, a second subregion image belonging to a tumor enhancement region and a third subregion image belonging to a peritumoral edema region from image samples of the tumor region; and
performing feature extraction on the first subregion image, the second subregion image and the third subregion image, respectively, to obtain the plurality of radiomics features.

3. The method according to claim 1, wherein acquiring the plurality of radiomics features of the tumor region comprises:
acquiring a plurality of types of image samples of the tumor region, wherein the plurality of types comprise a T1-weighted type, a T2-weighted type, a contrast-enhanced T1-weighted type and a T2 fluid-attenuated inversion recovery type;
performing feature extraction on each of the plurality of types of image samples, respectively; and
combining the radiomics features respectively corresponding to each of the plurality of types of extracted image samples to obtain the plurality of radiomics features.

4. The method according to claim 1, wherein the tumor region is a glioma region in a brain, and the method further comprises:
determining position information corresponding to the glioma region based on an image sample of the glioma region;
acquiring position features corresponding to the position information; wherein the position information comprises a brain region to which the glioma region belongs, and/or position coordinates of the glioma region in the brain; and
constructing the training samples based on the plurality of radiomics feature samples and the plurality of voxel feature samples comprises:
constructing the training samples based on the position features, the plurality of radiomics feature samples and the plurality of voxel feature samples.

5. The method according to claim 1, wherein the first screening factor comprises the expression category label and a tumor grading label of the tumor region; and screening the plurality of radiomics features based on the first screening factor to obtain the plurality of radiomics feature samples comprises:
screening the plurality of radiomics features based on a first relation value between each of the plurality of radiomics features and the expression category label to obtain a plurality of first radiomics features; wherein the first relation value is configured to represent a degree of correlation between each of the plurality of radiomics features and mutation of the target gene;
screening the plurality of radiomics features based on a second relation value between each of the plurality of radiomics features and the tumor grading label to obtain a plurality of second radiomics features; wherein the second relation value is configured to represent a degree of correlation between each of the plurality of radiomics features and a tumor grade; and
de-duplicating the plurality of first radiomics features and the plurality of second radiomics features to obtain the plurality of radiomics feature samples.

6. The method according to claim 1, wherein a plurality of sample objects are comprised, and the method further comprises:
for all the plurality of radiomics features comprised by all the sample objects, screening all the plurality of radiomics features based on a third screening factor to obtain complementary radiomics feature samples; wherein the third screening factor comprises clinical data respectively corresponding to the plurality of sample objects; and
constructing the training samples based on the plurality of radiomics feature samples and the plurality of voxel feature samples comprises:
constructing the training samples based on the plurality of radiomics feature samples, the plurality of voxel feature samples, and the plurality of complementary radiomics feature samples.

7. The method according to claim 6, wherein for all radiomics features comprised by all the sample objects, screening all the plurality of radiomics features based on the third screening factor to obtain the complementary radiomics feature samples comprises:
acquiring a radiomics feature matrix and a clinical data matrix; wherein the radiomics feature matrix comprises the plurality of radiomics features respectively corresponding to the plurality of sample objects, and the clinical data matrix comprises the clinical data respectively corresponding to the plurality of sample objects;
acquiring a mutual information coefficient matrix based on the radiomics feature matrix and the clinical data matrix, wherein the mutual information coefficient matrix comprises a mutual information coefficient between each of the plurality of radiomics features and the clinical data, and the mutual information coefficient is configured to represent a degree of correlation between each of the plurality of radiomics features and the clinical data; and
screening, based on the mutual information coefficient matrix, all the plurality of radiomics features comprised by the radiomics feature matrix to obtain the plurality of complementary radiomics feature samples.

8. The method according to claim 1, wherein the second screening factor comprises the expression category label, and screening the plurality of voxel features based on the second screening factor to obtain the plurality of voxel feature samples comprises:
acquiring a variance of each of the plurality of voxel features, and retaining voxel features of which variances are greater than a first variance threshold to obtain a plurality of candidate voxel features; and
taking the expression category label as a predication label and taking the plurality of candidate voxel features as inputs, screening the plurality of voxel feature samples from the plurality of candidate voxel features by using a linear regression model.

9. The method according to claim 1, wherein before screening the plurality of radiomics features based on the first screening factor to obtain the plurality of radiomics feature samples, the method further comprises:
determining a variance corresponding to each of the plurality of the radiomics features, and retaining radiomics features of which variances are greater than a second variance threshold to obtain a plurality of candidate radiomics features; and
screening the plurality of radiomics features based on the first screening factor to obtain the plurality of radiomics feature samples comprises:
screening the plurality of candidate radiomics features based on the first screening factor to obtain the plurality of radiomics feature samples.

10. The method according to claim 1, wherein acquiring the plurality of radiomics features of the tumor region comprises:
acquiring wavelet images and laplacian of gaussian (LoG) images of the image samples of the tumor region;
performing multi-scale feature extraction on the image samples of the tumor region, the wavelet images and the LoG images, respectively, to obtain first-order statistics features, texture features and morphological features of the tumor region; and
combining the first-order statistics features, the texture features and the morphological features of the tumor region to obtain the plurality of radiomics features.

11. The method according to claim 1, wherein training the preset model by taking the training samples as the inputs to obtain the classification model comprises:
inputting the training samples to the classification model to obtain a predicted expression category, outputted by the classification model, of a telomerase reverse tranase (TERT) gene;
determining a loss value of the classification model based on the predicted expression category and the expression category label;
updating parameters of the classification model based on the loss value; and
taking a classification model satisfying a training ending condition as the classification model, wherein the training ending condition is that the classification model converges or reaches a time quantity of preset updating.

12. A method for determining an expression category of a target gene, comprising:
acquiring a plurality of target radiomics features and a plurality of target voxel features of a tumor region of a to-be-tested object;
inputting the plurality of target radiomics features and the plurality of target voxel features to the classification model, wherein the classification model is obtained according to the method for acquiring the classification model according to claim 1; and
determining an expression category of a target gene of the to-be-tested object based on an output of the classification model.

13. The method according to claim 12, wherein after acquiring the plurality of target radiomics features and the plurality of target voxel features of the tumor region of the to-be-tested object, the method further comprises:
determining a variance corresponding to each of the plurality of target voxel features, and retaining target voxel features of which variances are greater than a first variance threshold; and
determining a variance corresponding to each of the plurality of target radiomics features, and retaining target radiomics features of which variances are greater than a second variance threshold;
inputting the plurality of target radiomics features and the plurality of target voxel features to the classification model comprises:
inputting the retained target radiomics features and the retained target voxel features to the classification model.

14. The method according to claim 12, wherein after acquiring the plurality of target radiomics features and the plurality of target voxel features of the tumor region of the to-be-tested object, the method further comprises:
acquiring a fourth screening factor corresponding to the to-be-tested object, wherein the fourth screening factor comprises clinical data and/or tumor grading data of the to-be-tested object;
screening the plurality of target radiomics features based on the fourth screening factor; and
inputting the plurality of target radiomics features and the plurality of target voxel features to the classification model comprises:
inputting the plurality of screened target radiomics features and the plurality of screened target voxel features to the classification model.

15. The method according to claim 14, wherein the fourth screening factor comprises the clinical data and the tumor grading data; and screening the plurality of target radiomics features based on the fourth screening factor comprises:
determining a third relation value between each of the target radiomics features and a tumor grading label and a mutual information coefficient between each of the plurality of target radiomics features and the clinical data;
screening the plurality of target radiomics features based on the third relation value, and screening the plurality of target radiomics features based on the mutual information coefficient; and
de-duplicating the target radiomics features screened based on the third relation value and the target radiomics features screened based on the mutual information coefficient to obtain the screened target radiomics features.

16. (canceled)

17. (canceled)

18. An electronic device, comprising a memory, a processor, and a computer program stored in the memory and capable of running on the processor, wherein the processor, when being executed, implements the method for acquiring the classification model according to claim 1.

19. A computer-readable storage medium, wherein a computer program stored thereon enables a processor, when being executed, to implement the method for acquiring the classification model according to claim 1.

20. An electronic device, comprising a memory, a processor, and a computer program stored in the memory and capable of running on the processor, wherein the processor, when being executed, implements the method for determining the expression category of the target gene according to claim 12.

21. A computer-readable storage medium, wherein a computer program stored thereon enables a processor, when being executed, to implement the method for determining the expression category of the target gene according to claim 12.

22. The method according to claim 2, wherein acquiring the plurality of radiomics features of the tumor region comprises:
acquiring a plurality of types of image samples of the tumor region, wherein the plurality of types comprise a T1-weighted type, a T2-weighted type, a contrast-enhanced T1-weighted type and a T2 fluid-attenuated inversion recovery type;
performing feature extraction on each of the plurality of types of image samples, respectively; and
combining the radiomics features respectively corresponding to each of the plurality of types of extracted image samples to obtain the plurality of radiomics features.
